(19) United States
(12) Patent Application Publication
(10) Pub. No.: US 2008/0220102 A1
(43) Pub. Date: Sep. 11, 2008

(54) Solubilsation Products of an Active Ingredient Extract

(76) Inventor: Dariush Behnam, Rossdorf (DE)

Correspondence Address: JACOBSON HOLMAN PLLC, 400 SEVENTH STREET N.W., SUITE 600, WASHINGTON, DC, 20004, US

(21) Appl. No.: 11/988,379

(22) PCT Filed: Jul. 7, 2006
(86) PCT No.: PCT/EP2006/006655

§ 371 Date: Jan. 7, 2008

(30) Foreign Application Priority Data
Jul. 8, 2005 (DE) 10 2005 032 352.9
Nov. 24, 2005 (DE) 10 2005 056 381.3
May 24, 2006 (DE) 10 2006 024 911.9

(51) Int. Cl. A61K 36/38; A61K 36/00; A61K 31/352

(52) U.S. Cl. 424/730; 424/725; 514/456

(57) ABSTRACT

The invention relates to a solubilizate of a phytoextract, which contains a physiological or pharmaceutical active ingredient, which solubilizate contains about 10% by weight to about 30% by weight of the phytoextract, about 4% by weight to about 35% by weight of water, which can be deionised, demineralised or distilled once or many times, and from about 55% by weight to about 75% by weight of an emulsifier having an HLB-value that ranges between 9-16, based on the solubilizate, which is equal to 100% by weight.

In the age of “Functional food”, it is no wonder that increasingly more nutritional physiologists and experts in the food industry, and also scholars from academic circles are interested in the active ingredients and nutrients present in plants. This interest is attributed to reasons related to consumer psychology in most cases.

Synthetically produced pure active ingredients require consistent maintenance of quality and, owing to the small volume, relatively uncomplicated and less efforts for the formulation to incorporate these substances into food. However, such highly concentrated synthetic products represent problems or barriers with regard to the food law.

Furthermore, the majority of consumers reject chemically produced food additives. Natural nutrients and active ingredients in plants, such as vegetables and fruits, are increasingly gaining acceptance among consumers and nutritional physiologists alike who are not constrained by the economy. In order to utilize this acceptance economically, the producers of food and cosmetics seek after e.g. plant extracts having high concentration of nutrients and active ingredients. New processes have been developed with a view to reduce the portion of undesirable concomitant substances in such extracts. While, in doing so, the natural food character of the extract is being increasingly removed in order to meet the requirements of the food law, the limits of economically justifiable technical feasibility have also been reached.

At the same time, the incorporation of such extracts into food poses another problem. The high amount and number of concomitant substances, which are irrelevant in terms of nutritional physiology or are undesirable to a certain extent, necessitate the use of large amounts of other additives (technological aids) in formulations in order to be able to incorporate the extract homogeneously and stably into the respective end products. However, such formulations have several disadvantages such as economic inefficiency, unpleasant sensory properties or insufficient long-term stability.

Furthermore, most of the interesting nutrients and active ingredients are lipophilic and thus poorly bioavailable to the human organism. Plant-based nutrients and active ingredients that are incorporated into cosmetics exhibit only a very moderate penetration into the skin. Since the extracts have only a relatively small concentration of nutrients and active ingredients, the low resorption of the lipophilic substances contained in the extracts casts the use of such extracts into doubt from the economic point of view.

The object underlying the invention is to process active ingredients, which are present in plants and have physiological or medicinal significance in such a way that they are more easily compatible with the human body even when present in higher concentrations.

For this purpose, the invention provides a solubilizate of a phytoextract containing the active ingredient, which solubilizate contains from about 10% by weight to about 30% by weight of the phytoextract, about 4% by weight to about 35% by weight of water, which can be deionized, demineralized or distilled once or many times, and from about 55% by weight to about 75% by weight of an emulsifier having an HLB-value ranging between 9-16, based on the solubilizate, which is equal to 100% by weight. The invention utilizes the fact that the active ingredient is contained with its natural concomitant substances and in natural concentrations, for example, in the commercially available phytoextract. Even if the active ingredient and its concomitant substances may split into several different micelles, when the micelles reaching the intestine empty out their content into the gut lumen, the latter receives not only the active ingredient but also all concomitant substances in natural concentration in the time average. The weight ratios specified have been selected such that the solubilizate of the invention is clear at body temperature, that is, at 37° C. and thus free from non-micellated portions and in a sufficiently liquid state.

For some phytoextracts, it is recommended, that the solubilizate of the invention contain a light oil, which comprises mainly medium-chain triglycerides, for example, Mygliol 812 or Delios VK kosher, in a proportion ranging from about 5% by weight to about 6% by weight. The oil prevents or complicates the precipitation of substances or substance particles from the solubilizate according to the invention.

The use of non-ionic, micelle-forming emulsifiers, primarily polysorbates, especially polysorbate 80 and/or polysorbate 20 as emulsifiers has proved to be useful. These emulsifiers are not only compatible with the requirements of the food law but also harmless in terms of their sensory properties. For protecting the active ingredients and concomitant substances from oxidation, it is recommended that the solubilizate of the invention contain one or more antioxidants such as, for example, ascorbic acid and/or a tocopherol, preferably a mixed tocopherol in a proportion ranging from about 4% by weight to about 6% by weight.

Furthermore, for obtaining a sediment-free micellization, it is advantageous in many cases if the weight ratio of phytoextract to water ranges from about 1.0 to about 3.0 and the weight ratio of phytoextract to polysorbate ranges from about 1.0 to about 6.0 in the solubilizate of the invention.

For a solubilizate containing about 10% reseda extract, the invention suggests, for example, that the solubilizate contain from about 15% by weight to about 30% by weight of water, about 10% by weight of reseda extract, and from about 60% by weight to about 75% by weight of polysorbate 80.

For a solubilizate containing about 10% by weight of St. John's wort extract, the invention suggests that the solubilizate contain from about 20% by weight to about 35% by weight of water, about 10% by weight of St. John's wort extract, and from about 55% by weight to about 70% by weight of polysorbate 80.

For a solubilizate containing about 10% by weight of tarragon extract, the invention suggests that the solubilizate contain from about 15% by weight to about 30% by weight of water, about 10% by weight of tarragon extract, and from about 60% by weight to about 75% by weight of polysorbate 80.

For a fat-soluble and water-soluble solubilizate containing about 12% hydroxytyrosol, the invention suggests by way of example that the solubilizate contain about 4% by weight of water, about 30% by weight of a hydroxytyrosol extract, which contains about 40% (based on the extract, which is equal to 100%) of hydroxytyrosol, about 58% by weight of polysorbate 80, about 4% by weight of a mixed tocopherol, and about 4% by weight of ascorbic acid.

For a fat-soluble and water soluble solubilizate containing about 15% rooibos extract, the invention suggests by way of example that the solubilizate contain about 15% by weight of water as distilled water, about 15% by weight of rooibos extract, about 60% by weight of polysorbate 80, about 5% by weight of a light oil containing mainly medium-chain triglycerides, and about 5% by weight of ascorbic acid.

For a fat-soluble and water-soluble solubilizate containing about 14% polyphenol, the invention suggests that the solubilizate contain about 14% by weight of water, about 14% by weight of a polyphenol extract, which predominantly contains tannin, gallic acid, and catechine as tanning agents, from about 30% by weight to about 33% by weight of polysorbate 20, from about 30% by weight to about 33% by weight of polysorbate 80, about 6% by weight of a light oil containing mainly medium-chain triglycerides, and from about 0% by weight to about 6% by weight of ascorbic acid.

For a rosemary extract-solubilizate, the invention suggests that the solubilizate contain from about 5% by weight to about 15% by weight of water, from about 15% by weight to about 20% by weight of a rosemary extract, which contains the antioxidant camosic acid, from about 50% by weight to about 75% by weight of polysorbate 80, from about 0% by weight to about 10% by weight of a light oil containing medium-chain triglycerides, from about 0% by weight to about 1.5% by weight of a mixed tocopherol, and from about 4% by weight to about 5% by weight of ascorbic acid.

Finally, for an olibanum-solubilizate, the invention suggests that the solubilizate contain about 24% by weight of water, about 12% by weight of an olibanum extract, which contains about 85% (based on the extract, which is equal to 100%) of boswellic acid, and about 64% by weight of polysorbate 20.

The solubilizates of the invention can be easily incorporated into cosmetic products such as ointments, pharmaceutical preparations, food preparations and food supplements and can be added to non-alcoholic drinks such as fruit juices and mineral water without any additional intermediate steps.

In a method suggested by the invention for producing solubilizates, from about 10% by weight to about 30% by weight of a phytoextract is mixed with from about 4% by weight to about 30% by weight of water, which is preferably deionized and/or demineralized and/or distilled once or many times, under mild heat of, for example, from about 40° C. to about 50° C. and the non-ionic and micelle-forming emulsifier, which has an HLB-value ranging from 9 to 16 and is heated to about 50°, is mixed into this mixture. The composition obtained is heated well, for example, to about 90° C. and homogenized until the fat-soluble and water-soluble solubilizate is obtained.

If the quantity of solubilizate, which contains as much active ingredient as is required to meet the daily human requirement, is dissolved in 1-liter of drinking water, the drink obtained therefrom indicates a turbidity of about 5.0 on a scale of from 0 to 2000 of a standard turbidimeter.

The following extracts are relevant to the invention.

### 1. Flavanoid Extract

Flavanoids, such as e.g. flavone “luteolin” C₁₅H₁₀)₆ or luteolin-7-glucoside C₂₁H₂₀O₁₁, which is contained in reseda luteola (dyer's rocket, weld), artichokes, capsicum or maidenhair-tree leaves, or can be educed therefrom by various methods is considered to be an extremely effective substance to prevent the formation of cholesterol in blood and actively promotes fat digestion. Flavanoids are polyphenols that are most frequently found in food.

It is extremely difficult to incorporate the extracts mentioned above into the respective end products in a homogeneous and stable manner and the substance “luteolin” present therein is both poorly bioavailable and unstable. The solubilization of this extract reduces these problems considerably.

### 2. Hypericum Extract

In addition to flavanoids such as, rutin, hyperoside, quercitrin, and quercitin, an extract derived from St. John's wort (Hypericum Perforatum) contains naphthodianthrone pseudohypercin, hepericin, and hyperforin.

Since generations, St. John's wort is considered to be a reliable and effective medicinal plant for the treatment and prevention of neurodegenerative disorders. This effect is mainly attributed to the substances of hypericin and hyperforin contained therein.

It is difficult to incorporate this extract as well into the respective end products and the active ingredients present therein are extremely unstable and poorly bioavailable. The solubilization of this extract enables easy incorporation into the end products, stabilizes the active ingredients, and results in significantly higher bioavailability and penetration.

3. Tarragon Extract (Artemisia dracunculus)

This plant originating in the South Russia and the Mongolian steppes contains inter alia high amounts of potassium, ascorbic acid, magnesium, vitamin A, and iodine. Tarragon is used inter alia as a medicinal plant in connection with rheumatic ailments and scurvy. In addition, tarragon is believed to provide relief in
diminished renal functioning,
dyspepsia,
anorexia,
dropsy, and
muscular cramps

Tarragon also promotes the formation of gastric juices.

The problems enumerated in items 1) and 2) above also apply to this extract. These problems can be significantly attenuated by a solubilization of the extract.

The invention will be explained below with reference to some exemplary embodiments.

### EXEMPLARY EMBODIMENT 1

Solubilizate containing 10% by weight of reseda extract

| Name of raw | Manufacturer/ | Batch number | |
|---|---|---|---|
| material | supplier | of the raw material | Formulation in % |
| Product water | | | 30.00% |
| Reseda extract | | Res. 07404.02 | 10.00% |
| Polysorbate 80 | Cognis | GR 32379104 | 60.00% |
| Product water | | | 25.00% |
| Reseda extract | | Res. 07404.02 | 10.00% |
| Polysorbate 80 | Cognis | GR 32379104 | 65.00% |
| Product water | | | 20.00% |
| Reseda extract | | Res. 07404.02 | 10.00% |
| Polysorbate 80 | Cognis | GR 32379104 | 70.00% |
| Product water | | | 15.00% |
| Reseda extract | | Res. 07404.02 | 10.00% |
| Polysorbate 80 | Cognis | GR 32379104 | 75.00% |

Mix reseda extract with product water (at 50° C.). Heat polysorbate to 50° C. and incorporate into the mixture composed of extract and water with stirring. Heat again thoroughly (approx. 90° C.) and homogenize.

### EXEMPLARY EMBODIMENT 2

Solubilizate containing 10% by weight of St. John's wort extract

| Name of raw | Manufacturer/ | Batch number | Formulation |
|---|---|---|---|
| material | supplier | of the raw material | in % |
| Product water | | | 35.00% |
| Dry extract of St. | Euromed S.A. | Lot 158041 | 10.00% |
| John's wort | | | |
| Polysorbate 80 | Cognis | GR 32379104 | 55.00% |
| Product water | | | 30.00% |
| Dry extract of St. | Euromed S.A. | Lot 158041 | 10.00% |
| John's wort | | | |
| Polysorbate 80 | Cognis | GR 32379104 | 60.00% |
| Product water | | | 25.00% |
| Dry extract of St. | Euromed S.A. | Lot 158041 | 10.00% |
| John's wort | | | |
| Polysorbate 80 | Cognis | GR 32379104 | 65.00% |
| Product water | | | 20.00% |
| Dry extract of St. | Euromed S.A. | Lot 158041 | 10.00% |
| John's wort | | | |
| Polysorbate 80 | Cognis | GR 32379104 | 70.00% |

Mix hyperforin with product water (at approx. 45° C.). Heat polysorbate to 50° C. and incorporate into the mixture composed of extract and water with stirring. Heat again thoroughly (approx. 90° C.) and homogenize.

### EXEMPLARY EMBODIMENT 3

Solubilizate containing 10% by weight of tarragon extract

| Name of raw | Manufacturer/ | Batch number | Formulation |
|---|---|---|---|
| material | supplier | of the raw material | in % |
| Product water | | | 30.00% |
| Tarragon extract | Degussa | Lot 04 120 379 | 10.00% |
| Polysorbate 80 | Cognis | GR 32379104 | 60.00% |
| Product water | | | 25.00% |
| Tarragon extract | Degussa | Lot 04 120 379 | 10.00% |
| Polysorbate 80 | Cognis | GR 32379104 | 65.00% |
| Product water | | | 20.00% |
| Tarragon extract | Degussa | Lot 04 120 379 | 10.00% |
| Polysorbate 80 | Cognis | GR 32379104 | 70.00% |
| Product water | | | 15.00% |
| Tarragon extract | Degussa | Lot 04 120 379 | 10.00% |
| Polysorbate 80 | Cognis | GR 32379104 | 75.00% |

Mix tarragon extract with product water (at approx. 50° C.). Heat polysorbate to 50° C. and incorporate into the mixture composed of extract and water with stirring. Heat again thoroughly (approx. 90° C.) and homogenize. The solubilizates obtained from the exemplary embodiments described above are fat-soluble and water-soluble, and stable against thermal influencing and stomach acids. The turbidity (based on the respective daily requirement in e.g. a 1 liter drink) on a scale of 0-2000 is approx. 5.0.

| | |
|---|---|
| Description: | Fat-soluble and water-soluble, 12% hydroxytyrosol- |
| | solubilizate with 4% ascorbic acid and 4% mixed |
| | tocopherol |
| Ingredients: | |
| 40 g | Ascorbic acid (Takeda); BASF (AGT material no.: |
| | 10710/005) |
| 40 g | Product water; (AGT material no.: 10180) |
| 300 g | Hytolive ® 2 (contains 40% hydroxytyrosol); |
| | I + D (AGT material no.: 10308/085) |
| 40 g | Decanox MTS-70 IP (mixed tocopherol); ADM/Parmentier |
| | (AGT material no.: 10727/001) |
| 580 g | Lamesorb SMO 20 (polysorbate 80); Cognis (AGT |
| | material no.: 10530/016) |

### Procedure:

Mix ascorbic acid and water, warm it slightly within a controlled temperature range (max. 85° C.) until the ascorbic acid dissolves completely forming a clear solution.
Add mixed tocopherol to the ascorbic acid-water mixture with stirring (83-87° C.).
Meter Hytolive® 2 with stirring. Homogenize thoroughly (83-87° C.).
Add polysorbate 80 with stirring. Homogenize well (83-87° C.).
Let cool slightly and fill at approx. 60° C. or lower.

Appearance:

Dark brown, viscous

Storage Conditions:

Dark and cool (<25°)

| | |
|---|---|
| Description: | Water-soluble and fat-soluble, 15% rooibos extract- |
| | solubilizate with 5% ascorbic acid |
| Ingredients: | |
| 50 g | Ascorbic acid; BASF (AGT material no.: 10710/005) |
| 150 g | Distilled water; (AGT material no.: 10180) |
| 50 g | Delios VK kosher (MCT oil), Cognis (AGT material no.: |
| | 10460/016) |
| 150 g | Rooibos extract; powder; (AGT material no.: 10581/084) |
| 600 g | Polysorbate 80; Lamesorb SMO 20; Cognis (AGT |
| | material no.: 10530/016) |

### Procedure:

Mix ascorbic acid and water, warm it slightly within a controlled temperature range (max. 80° C.) until the ascorbic acid dissolves completely forming a clear solution.
Incorporate MCT oil into the ascorbic acid-water mixture and let cool to 38-42° C.
Add Rooibos extract to the mixture (temperature must not exceed 40° C.) and homogenize well.
Slowly add polysorbate 80 to the mixture with stirring. Heat within a controlled temperature ranging between 83-87° C. stirring constantly. Let stand until everything is homogenized well and tempered.
Let cool to <35° and fill.

Appearance:

Dark brown-black, viscous gel-like

Storage Conditions:

Dark, at room temperature (<25°)

| | |
|---|---|
| Description: | Water-soluble and fat-soluble, 14% polyphenol |
| | (Bio-TAN)/6% ascorbic acid-solubilizate |
| Ingredients: | |
| 140 g | product water; (AGT material no.: 10180) |
| 60 g | Ascorbic acid; BASF (AGT material no.: 10710/005) |
| 140 g | Polyphenol extract; sample supplied by Dr. Martin |
| | Schmitt, Bad Kreuznach (AGT material no.: 10540/087) |
| 60 g | Delios VK kosher (MCT oil); Cognis (AGT material |
| | no.: 10460/016) |
| 300 g | Lamesorb SML 20 (Polysorbate 20); Cognis (AGT |
| | material no.: 10520/016) |
| 300 g | Lamesorb SMO 20 (Polysorbate 80); Cognis (AGT |
| | material no.: 10530/016) |

### Procedure:

Mix product water and ascorbic acid and heat to 80° C. with stirring until a clear solution is obtained.
With stirring, add polyphenol extract to the ascorbic acid-water mixture and heat to 93-97° C.
As soon as a clear solution is obtained, incorporate the MCT oil into the mixture with stirring, maintain temperature (93-97° C.)
Mix the two polysorbates and slowly incorporate into the polyphenol extract/ascorbic acid/water/MCT oil mixture with stirring.
Homogenize well. Maintain the temperature (93-97° C.) until a clear solubilizate is obtained.
Let cool and fill at <=60° C.

### Appearance:

Yellow, viscous, transparent

### Storage Conditions:

Dark and cool (<25°)

| | |
|---|---|
| Description: | Water-soluble and fat-soluble 15% rosemary extract- |
| | solubilizate with ascorbic acid |
| Ingredients: | |
| 50 g | Ascorbic acid; BASF (AGT material no.: 10710/005) |
| 50 g | distilled water; (AGT material no.: 10180) |
| 750 g | Polysorbate 80; Lamesorb SMO 20; Cognis (AGT |
| | material no.: 10530/016) |
| 150 g | Rosemary extract; oily; Arjuna Natural Extracts Ltd. |
| | (AGT material no.: 1059/081) |

### Procedure:

Mix ascorbic acid and water, warm it slightly within a controlled temperature range (max. 75° C.) until the ascorbic acid dissolves completely forming a clear solution.
Incorporate polysorbate 80 into the ascorbic acid/water mixture with stirring, homogenize thoroughly (85±2° C.)
Add rosemary extract with stirring and homogenize thoroughly (85±2° C.)
Let cool to <35° C. and bottle.

### Appearance:

brown, viscous

### Storage Conditions:

Dark, at room temperature (<25°)

| | |
|---|---|
| Description: | Water-soluble and fat-soluble, 14% polyphenol |
| | (Bio-TAN)-solubilizate |
| Ingredients: | |
| 140 g | product water; (AGT-Material no.: 10180) |
| 140 g | Polyphenol extract; sample supplied by Dr. Martin |
| | Schmitt, Bad Kreuznach (AGT material no.: 10540/087) |
| 60 g | Delios VK kosher (MCT oil); Cognis (AGT material |
| | no.: 10460/016) |
| 330 g | Lamesorb SML 20 (Polysorbate 20); Cognis (AGT |
| | material no.: 10520/016) |
| 330 g | Lamesorb SMO 20 (Polysorbate 80); Cognis (AGT |
| | material no.: 10530/016) |

### Procedure:

Heat product water to 37-42° C.
With stirring, add polyphenol extract to the water and heat to 93-97° C.
As soon as a clear solution is obtained, incorporate the MCT oil into the mixture with stirring, maintain temperature (93-97° C.)
Mix the two polysorbates and slowly incorporate into the polyphenol extract/water/MCT oil mixture with stirring.
Homogenize well. Maintain the temperature (93-97° C.) until a clear solubilizate is obtained.
Let cool and fill at <=60° C.

### Appearance:

Yellow, viscous, transparent

### Storage Conditions:

Dark and cool (<25°)

| | |
|---|---|
| Description: | Water-soluble and fat-soluble 15% rosemary extract- |
| | solubilizate with 5% ascorbic acid |
| Ingredients: | |
| 50 g | Ascorbic acid; BASF (AGT material no.: 10710/005) |
| 150 g | Distilled water; (AGT material no.: 10180) |
| 100 g | Delios VK kosher (MCT oil); Cognis (AGT material no.: |
| | 10460/016) |
| 150 g | Rosemary extract; powder; (AGT material no.: 10592/084) |
| 550 g | Polysorbate 80; Lamesorb SMO 20; Cognis (AGT |
| | material no.: 10530/016) |

### Procedure:

Mix ascorbic acid and water, warm it slightly within a controlled temperature range (max. 80° C.) until the ascorbic acid dissolves completely forming a clear solution.
Sieve rosemary extract powder and mix it into the MCT oil, dissolve and heat slowly (max. 75° C.)
Incorporate this mixture of rosemary extract and MCT oil into the water-ascorbic acid mixture with stirring (maintain temperature at max. 75° C.)
Add polysorbate 80 to the remaining mixture with stirring, stir well and heat within a controlled temperature ranging between 83-87° C., and let stand for some time until everything is homogenized well and tempered.
Let cool to <35° C. and fill.

### Appearance:

Dark brown-black, viscous, typical smell of Rosemary

| | |
|---|---|
| Description: | NovaSOL ® rosemary water-soluble and |
| | fat-soluble 15% rosemary extract-solubilizate with 4% |
| | ascorbic acid and 1% mixed tocopherol |
| Ingredients: | |
| 40 g | Ascorbic acid; BASF (AGT material no.: 10710/005) |
| 150 g | product water; (AGT material no.: 10180) |
| 86 g | Delios VK kosher (MCT oil); Cognis (AGT material |
| | no.: 10460/016) |
| 150 g | Rosemary extract; powder; (AGT material no.: 10592/089) |
| 14 g | Decanox MTS 70-IP (mixed tocopherol); ADM/Parmentier |
| | (AGT material no. 10727/001) (corresponds to 1% effective |
| | mixed tocopherol) |
| 560 g | Polysorbate 80; Lamesorb SMO 20; Cognis (AGT |
| | material no.: 10530/016) |

### Procedure:

Mix ascorbic acid and water, warm it slightly within a controlled temperature range (max. 80° C.) until the ascorbic acid dissolves completely forming a clear solution.
Add MCT oil to the ascorbic acid/water mixture with stirring (max. 75° C.)
Sieve rosemary extract and incorporate slowly into the ascorbic acid/water/MCT oil mixture with stirring (maintain temperature at max. 75° C.)
Add mixed tocopherol with stirring, maintain temperature.
Add polysorbate 80 into the remaining mixture with stirring. Mix thoroughly and heat within a controlled temperature ranging between 83-87° C. Let stand for some time until everything is homogenized and tempered well.
Let cool to <35° C. and fill.

### Appearance:

Dark brown-black, viscous, typical smell of rosemary

### Storage Conditions:

Dark, at room temperature (<25°)

| | |
|---|---|
| Description: | Water-soluble and fat-soluble 20% rosemary extract- |
| | solubilizate with ascorbic acid |
| Ingredients: | |
| 50 g | Ascorbic acid; BASF (AGT material no.: 10710/005) |
| 50 g | distilled water; (AGT material no.: 10180) |
| 700 g | Polysorbate 80; Lamesorb SMO 20; Cognis (AGT |
| | material no.: 10530/016) |
| 200 g | Rosemary extract; oily; Arjuna Natural Extracts Ltd. |
| | (AGT material no.: 1059/081) |

### Procedure:

Mix ascorbic acid and water, warm it slightly within a controlled temperature range (max. 75° C.) until the ascorbic acid dissolves completely forming a clear solution.
Incorporate polysorbate 80 into the ascorbic acid/water mixture with stirring, homogenize thoroughly (85±2° C.)
Add rosemary extract with stirring and homogenize thoroughly (85±2° C.)
Let cool to <35° C. and fill.

### Appearance:

Brown, viscous

### Storage Conditions:

Dark, at room temperature (<25°)

### EXEMPLARY EMBODIMENT

Ingredients:

| | |
|---|---|
| 118 g | Olibanum extract 85%, powder (= 85% boswellic acid); |
| | Pfannenschmidt (AGT material no..: 10111/029), cf. page 4 |
| 242 g | Product water; (AGT material no.: 10180) |
| 640 g | Lamesorb SML 20 (polysorbate 20); Cognis (AGT |
| | material no.: 10520/016) |

### Procedure:

Mix product water with the olibanum extract powder and heat (87-93° C.)
Maintaining temperature, slowly incorporate polysorbate 20 into the mixture and homogenize well.
There will be a strong foam formation—this can be ignored as long as a clear solubilizate is visible on the base of the container when drawing the sample.
Let cool and fill at approx. 50° C.

### Appearance:

Brownish, clear, viscous.

1. Solubilizate of a phytoextract, which contains a physiological or pharmaceutical active ingredient, which solubilizate contains from about 10% by weight to about 30% by weight of the phytoextract, about 4% by weight to about 35% by weight of water, which can be deionized, demineralized or distilled once or many times, and about 55% to about 75% by weight of an emulsifier having an HLB-value that ranges between 9-16, based on the solubilizate, which is equal to 100% by weight.

2. Solubilizate according to claim 1 containing a portion ranging from about 5% by weight to about 6% by weight of a light oil, which comprises mainly medium-chain triglycerides.

3. Solubilizate according to claim 1 containing a non-ionic, micelle-forming emulsifier, in particular one or more polysorbates, preferably polysorbate 20 and/or polysorbate 80.

4. Solubilizate according to claim 1 containing one or more antioxidants such as ascorbic acid or a tocopherol, preferably a mixed tocopherol.

5. Solubilizate according to claim 3, in which the weight ratio of phytoextract to water ranges from about 1.0 to about 3.0 and the weight ratio of phytoextract to polysorbate ranges from about 1.0 to about 6.0.

6. Solubilizate according to claim 1, which contains from about 15% by weight to about 30% by weight of water, about 10% by weight of reseda extract, and from about 60% by weight to about 75% by weight of polysorbate 80.

7. Solubilizate according to claim 1, which contains from about 20% by weight to about 35% by weight of water, about 10% by weight of St. John's wort extract, and from about 55% by weight to about 70% by weight of polysorbate 80.

8. Solubilizate according to claim 1, which contains from about 15% by weight to about 30% by weight of water, about 10% by weight of tarragon extract, and from about 60% by weight to about 75% by weight of polysorbate 80.

9. Solubilizate according to claim 1, which contains about 4% by weight of water, about 30% by weight of a hydroxytyrosol extract, about 58% by weight of polysorbate 80, about 4% by weight of a mixed tocopherol, and about 4% by weight ascorbic acid.

10. Solubilizate according to claim 1, which contains about 15% by weight of water, about 15% by weight of rooibos extract, about 60% by weight of polysorbate 80, about 5% by weight of a light oil containing medium-chain triglycerides, and about 5% by weight of ascorbic acid.

11. Solubilizate according to claim 1, which contains about 14% by weight of water, about 14% by weight of polyphenol extract, which predominantly contains tannin, gallic acid, and catechine, from about 30% by weight to about 33% by weight of polysorbate 20, from about 30% by weight to about 33% by weight of polysorbate 80, and from about 0% by weight to about 6% by weight of ascorbic acid.

12. Solubilizate according to claim 1, which contains from about 5% by weight to about 15% by weight of water, from about 15% by weight to about 20% by weight of a rosemary extract, which contains carnosic acid, from about 50% by weight to about 75% by weight of polysorbate 80, from about 0% by weight to about 10% by weight of a light oil containing medium-chain triglycerides, from about 0% by weight to about 1.5% by weight of a mixed tocopherol, and from about 4% by weight to about 5% by weight of ascorbic acid.

13. Solubilizate according to claim 1, which contains about 24% by weight of water, about 12% by weight of an olibanum extract, which contains about 85% (based on the extract, which is equal to 100%) of boswellic acid, and about 64% by weight of polysorbate 20.

14. Method for producing solubilizates according to claim 1, in which from about 10% by weight to about 30% by weight of a phytoextract is mixed with from about 4% by weight to about 30% by weight of water, which is preferably deionized or demineralized or distilled once or many times, under mild heat of, for example, from about 40° C. to about 50° C. and the emulsifier, which has an HLB-value ranging from 9 to 16 and is heated to about 50° C., is mixed into this mixture, the composition obtained is heated well, for example, to about 90° C. and homogenized until the fat-soluble and water-soluble solubilizate is obtained.

15. Use of a solubilizate according to claim 1 as an additive in cosmetic products such as, for example, ointments, pharmaceutical preparations, food preparations, and food supplements, and non-alcoholic drinks such as, for example, fruit juices and mineral water.
